(19) United States
(12) Patent Application Publication
(10) Pub. No.: US 2008/0257338 A1
(43) Pub. Date: Oct. 23, 2008

(54) Medicament Delivery Device Comprising a Flexible Edible Bag

(75) Inventor: Adrian Gee-Turner, London (GB)

Correspondence Address: BEYER WEAVER LLP, P.O. BOX 70250, OAKLAND, CA, 94612-0250, US

(73) Assignee: OMRON HEALTHCARE Co., Ltd.

(21) Appl. No.: 11/884,981

(22) PCT Filed: Feb. 24, 2006
(86) PCT No.: PCT/GB2006/000651

§ 371 Date: Aug. 22, 2007

(30) Foreign Application Priority Data
Feb. 25, 2005 (EP) 05251138.3

(51) Int. Cl. A61M 11/00

(52) U.S. Cl. 128/200.23

(57) ABSTRACT

This invention relates to devices, uses and methods for the delivery of a medicament in aerosol form. The proposed arrangement permits simple, cost-effective delivery (e.g. for mass immunisation programmes) which avoids contamination risk and/or disposal difficulties. Medicament in aerosol form is supplied either to the interior of an edible bag, or to the interior of a funnel which is lined with a liner, which bag or funnel is placed over a patient's nose and mouth to enable inhalation of the medicament. The liner may be edible, e.g. made of rice paper or food-based polymer. A nebuliser is used to generate the aerosol using a flow of compressed air. Both the bag and the funnel have a suitably sized opening for fitting over the patient's nose and mouth.

This invention relates to devices, uses and methods for the delivery of a medicament in aerosol form.

A number of medicaments are suitable for delivery to the lungs in the form of an aerosol. These include certain vaccines such as measles vaccine. Delivery of medicaments in the form of an aerosol has a number of advantages over other routes of administration. In the case of vaccination, aerosol delivery to the lungs avoids many disadvantages associated with conventional injection delivery, such as cost of needles, safe disposal of used needles and fear of injections, especially among children. These disadvantages are particularly problematic for large-scale immunization programmes in economically underdeveloped countries. Moreover, medical trials suggest that the use of aerosols result in a higher immunisation coverage and fewer side effects than subcutaneous injection. Delivery of medicaments in aerosol form is also preferred for many pulmonary conditions such as asthma.

Conventional delivery systems for medicaments in aerosol form generally comprise means to convert a liquid medicament into an aerosol, such as a nebuliser, together with means to direct the aerosol into a patient's nose or mouth such as a mask or a rigid container with a nozzle. The patient inhales the aerosol from the mask or chamber. Conventionally, the mask or chamber is re-usable and often has one or more valves. Due to the risk of contamination, re-use of such systems requires frequent, thorough cleaning. Moreover, the complexity of the construction leads to considerable cost. Particularly in the setting of a major immunisation programme, cost, disposal difficulties and likelihood of contamination constitute substantial disadvantages.

The present invention seeks to provide arrangements for delivery of a medicament in aerosol form which are sufficiently simple and cost-effective to be used in a mass immunisation programme.

At its most general, the present invention proposes that the medicament is supplied in aerosol form to the interior of either a bag of flexible edible material, or the interior of a funnel (or mask) which funnel is lined with flexible material. and In the case where a bag is used, once the interior of the bag is supplied with aerosol, it is then placed over the nose and/or mouth of the person to be immunised (hereinafter “a patient”). The patient then inhales the air, and hence the aerosol, from the bag thereby delivering the medicament to the patient.

In such an arrangement, the bag will need to have an opening of suitable size to fit over the nose and/or mouth of the patient, and this can easily be achieved by suitable shaping of the opening of the bag. The aerosol of medicament is introduced into the bag from e.g. a nebuliser, and the opening of the bag is then closed to hold the aerosol within the bag. Provided that the time between the supply of aerosol into the bag and the time the bag is placed over the nose and/or mouth of the patient is reasonably short, manual constriction of the opening of the bag should provide sufficient sealing for there to be enough medicament in the bag when it is placed over the nose and/or mouth of the patient for the patient to inhale a suitable dose.

In the case where a funnel is used, the interior of the funnel is lined with a flexible material to prevent direct contact between the patient and the interior of the funnel. A first open end of the funnel is placed over the nose and/or mouth of the patient, ensuring that any contact of any part of the patient's face or body with the interior of the funnel is made via the liner. In this way the liner acts as a barrier between the patient and the interior of the funnel, so the interior of the funnel is not contaminated by the patient. Aerosol is introduced to the interior of the funnel via a second open end of the funnel and the patient then inhales the air, and hence the aerosol, from the funnel thereby delivering medicament to the patient.

In such an arrangement, the first open end of the funnel will need to be of a suitable size and shape to fit over the nose and/or mouth of the patient.

Inhalation from and exhalation into the bag or funnel may be repeated to ensure that the patient inhales a suitably large dose of the medicament.

The present invention has several aspects. Thus, according to the first aspect, the present invention may provide a kit of parts for use in delivery of a medicament to a patient, comprising:

at least one bag of flexible material;

a container for said medicament; and

means for generating an aerosol of at least part of said medicament in said container for supply of said aerosol to the interior of the bag

According to the second aspect of the present invention there may be provided a medicament delivery device comprising a bag of flexible material, and a medicament in aerosol form in said bag, wherein said bag is closed but is openable to fit over the nose and/or mouth of a patient.

As the bag is preferably of edible material, it can be ingested readily by the patient, which provides a means of disposal of the bag and so prevents cross-contamination from one patient to another. To achieve such an edible bag, the bag may be made of rice paper or a food-derived polymer or cellulose, vegetable or fruit. Moreover, it is preferable that the bag is impregnated with vitamins and/or nutritional minerals, so that those vitamins and/or nutritional minerals may be absorbed by the patient when the bag is ingested. This permits the bag to serve a second purpose after the medicament has been ingested.

A further possibility is for the bag to be at least partially dissolved in water or other liquid to soften it prior to being eaten. Softening may tend to cause vitamins and/or nutritional minerals in the bag to dissolve in the water, so that the water can then be drunk.

According to a third aspect of the present invention there may be provided an edible bag for the production of a medicament delivery device for treatment of a vaccinable disease by supply of said medicament in aerosol form for inhalation by a patient the bag being of edible material and having an opening for fitting over the nose and/or mouth of a patient.

According to a fourth aspect of the present invention there may be provided a method of treatment of a patient, comprising:

introducing medicament in aerosol form into the hollow interior of a bag of flexible edible material, the bag having an opening for fitting over the nose and/or mouth of the patient through which opening the medicament is introduced into the bag;

temporarily closing the bag to retain the aerosol therein;

opening the bag and fitting the opening over the nose and/or mouth of the patient;

supplying the medicament to the patient by inhalation of the aerosol by the patient from the bag.

As mentioned above, the bag may be closed by manual constriction between the time in which the aerosol is supplied to the interior of the bag and the time at which the bag is placed over the nose and/or mouth of the user. To facilitate such manual constriction of the bag, the bag may have a neck of reduced width compared to the opening, so that the bag is splayed between the neck and the opening. In this way, the bag is closed by constriction at the neck to prevent loss of a significant amount of the aerosol, but the splaying of the bag enables it to be placed over the nose and/or mouth of the patient whilst the manual constriction is still present, so that there is no great loss of aerosol on release of the constriction. The neck also facilitates manual constriction of the bag onto e.g. a nozzle of the nebuliser which will supply the medicament in aerosol form.

It is convenient if the bag is supplied in the form of a sheet, e.g. of two-ply material with the plies sealed together to define the periphery of the bag. The sheet may be perforated in a shape conforming to the periphery of the bag so that a plurality of such sheets may be provided to a site where treatment of a plurality of patients is to be carried out, with the sheets being in a pad or block. Then, for each patient, a sheet is removed from the pad or block, perforations are broken to release the bag from the rest of the sheets, then medicament in aerosol form is supplied to the interior of the bag.

Another alternative is for such sheets or the bags themselves to be joined end-to-end and formed e.g. into a roll on a bobbin for storage and/or distribution. By perforating the joins between the bags/sheets it becomes possible to detach one bag/sheet from the roll, keeping the next bag/sheet clean until it is needed.

According to a fifth aspect of the present invention there may be provided a device for use in delivery of a medicament to a patient, comprising:

a funnel having a first open end and a second open end,

a liner of flexible material, said liner lining the interior of said funnel,

a container for said medicament,

an apparatus for generating an aerosol of at least part of said medicament in said container, and

a conduit arranged between said apparatus for generating an aerosol and said second open end of said funnel, thereby to permit said aerosol to be supplied from said apparatus to the interior of said funnel via said second open end, for inhalation by the patient via said first open end.

Preferably said conduit includes a branch channel, diverging from the conduit between said apparatus and said second open end of said funnel, which branch channel includes a filter. Accordingly, when the patient breathes out into the funnel, the filter filters expired breath to reduce the chance of contaminating a nurse administering the medicament to the patient.

After use of the device according to the fifth aspect of the present invention by a patient, the liner may be disposed of, leaving the funnel uncontaminated and re-usable by subsequent patients.

Preferably the conduit is made in one piece, and preferably it is made from a material comprising polyethylene. Furthermore, the conduit may be made from a material comprising k resin ABS.

Preferably said funnel is made of flexible material, e.g. silicon rubber. This has the advantage that the funnel is flexible to allow it to more easily mould to the contours of the face of the patient.

Preferably, the liner in said funnel also covers a rim of the first open end of said funnel. With the rim of said first open end of the funnel so covered, no part of the first end of the funnel may be contaminated by a patient. Preferably the rim of the first open end of said funnel is flanged, such that the rim is bent back on itself, and said liner envelops said flange. Movement of the liner relative to the funnel may be restricted by it being trapped by flanges and at the first open end and second open end of the funnel. This means that a barrier between a patient and the funnel is more likely maintained.

Preferably the liner of said kit of parts also covers a rim of the second open end of said funnel. With the rim of said second open end of the funnel so covered, no part of the second end of the funnel may be contaminated by a patient. Preferably the rim of the second open end of said funnel is flanged, such that the rim is bent back on itself, and said liner envelops said flange.

Preferably said apparatus for generating an aerosol is a nebuliser. More preferably, there is provided a compressor for driving said nebuliser. However, alternative means of generating an aerosol may be used, e.g. a spray of compressed gas having the medicament mixed therein. The compressor may have a battery and/or a power line terminating in a plug for connection to an A/C power source. Indeed, the battery may be re-chargeable from the A/C source to enable the nebuliser to be used at sites remote from A/C power sources. A solar power system may also be used to drive the compressor, as may a DC 12V power source.

It is also preferable that the liner is of edible material. This means that it can be ingested readily by the patient, which provides a means of disposal of the liner and so prevents cross-contamination from one patient to another. To achieve such an edible liner, the liner may be made of rice paper or a food-derived polymer or cellulose, vegetable or fruit. Moreover, it is preferable that the liner is impregnated with vitamins and/or nutritional minerals, so that those vitamins and/or nutritional minerals may be absorbed by the patient when the liner is ingested. This permits the liner to serve a second purpose after the medicament has been ingested.

A further possibility is for the liner to be at least partially dissolved in water or other liquid to soften it prior to being eaten. Softening may tend to cause vitamins and/or nutritional minerals in the liner to dissolve in the water, so that the water can then be drunk.

However, the liner is not limited to being made of edible material. The use of paper, even if not readily edible, is preferred because of cost considerations. However, plastics materials may be used although they then introduce the issue of disposal.

The diameter of a rim of one open end of the funnel is of course greater than that of the rim of the other open end of the funnel. Either the wider or narrower open end may act as the second open end of the funnel, leaving the other open end to act as the first open end, which is brought into contact with the nose and/or mouth of a patient. This has the advantage that the device may be assembled with the funnel orientated to present the narrower open end of the funnel to a patient, should the wider open end of the funnel be too broad to mould suitably to the mouth and nose of the patient, e.g. if the patient is an infant.

The passageway through the funnel may be closed by manual constriction, particularly between the time in which the aerosol is supplied to the interior of the funnel and the time at which the funnel is placed over the nose and/or mouth of the user. To facilitate such manual constriction of the funnel, the funnel preferably has a central portion of reduced diameter compared to the open ends. In this way, the funnel is closed by constriction between the open ends to prevent loss of a significant amount of the aerosol. This will allow medicament to build up in the funnel such that, once the constriction is removed and the funnel is placed over the nose and/or mouth of the patient, the patient is able to inhale a suitable dose from the interior of the funnel.

According to the sixth aspect of the present invention there may be provided a kit of parts for use in delivery of a medicament to a patient, comprising:

a funnel having a first open end and a second open end,

a liner of flexible material, said liner being adapted to line the interior of said funnel,

a container for said medicament,

an apparatus for generating an aerosol of at least part of said medicament in said container, said apparatus adapted to supply the aerosol to the interior of the funnel via said second open end, and

a conduit adapted to be connectable by a first end to said second open end of said funnel and connectable by a second end to said apparatus for generating an aerosol.

Preferably said conduit includes a branch channel, which branch channel branches from said conduit between said first and second ends of said conduit, and which branch channel includes a filter.

Preferably said apparatus for generating an aerosol is a nebuliser, and more preferably said nebuliser is driven by a compressor.

Either of the wider or narrower of the two open ends of said funnel is adapted to be connectable to said conduit, to thus form the second open end of the funnel.

It is preferred that the liner is part of a sheet, e.g. of two-ply material with the plies sealed together to define the periphery of the liner. The sheet may be perforated in a shape conforming to the periphery of the liner so that a plurality of such sheets may be provided to a site where treatment of a plurality of patients is to be carried out, with the sheets being in a pad or block. Then, for each patient, a sheet is removed from the pad or block, perforations are broken to release the liner from the rest of the sheets, the liner is inserted into the funnel of the device, the first open end of the funnel is introduced to the mouth of a patient and then medicament in aerosol form is supplied to the interior of the funnel via the second open end of the funnel. It is further preferred that said sheet is one of a plurality of identical sheets.

Another alternative is for such sheets or the liners themselves to be joined end-to-end and formed e.g. into a roll on a bobbin for storage and/or distribution. By perforating the joins between the liners/sheets it becomes possible to detach one liner/sheet from the roll, keeping the next liner/sheet clean until it is needed.

According to a seventh aspect of the present invention, there may be provided a method of assembling a device for delivery of a medicament to a patient, said device comprising:

a funnel having a first open end and a second open end,

a liner of flexible material, said liner being adapted to line the interior of said funnel,

a container for said medicament, and

an apparatus for generating an aerosol of at least part of said medicament in said container to supply the aerosol to the interior of the funnel via said second open end,

said method including the steps of:

arranging the liner in the funnel such that the liner lines the interior of the funnel,

connecting the second open end of said funnel to said apparatus for generating an aerosol such that they are in fluid communication, and

connecting said apparatus for generating an aerosol to said container such that they are in fluid communication.

Preferably the step of arranging the liner in the funnel such that the liner lines the interior of the funnel is carried out before the step of connecting the second open end of said funnel to said apparatus for generating an aerosol. However, these steps may be carried out in the reverse order.

Preferably the liner is of edible material. Preferably when said liner is arranged to line the interior of the funnel it also covers a rim of the first open end of said funnel. More preferably, when said liner is arranged to line the interior of the funnel it also covers a rim of said second open end of said funnel.

Preferably the rim of the first open end of said funnel is flanged, such that the rim is bent back on itself, and when said liner is arranged to line the interior of the funnel it also envelops said flange. More preferably, the rim of the second open end of said funnel is flanged, such that the rim is bent back on itself, and when said liner is arranged to line the interior of the funnel it also envelops said flange.

The step of connecting the second open end of said funnel to said apparatus for generating an aerosol may include the steps of:

connecting the second open end of said funnel to a first end of a conduit, and

connecting said apparatus for generating an aerosol to a second end of said conduit,

wherein said conduit includes a branch channel, which branches from said conduit between the first and second ends of said conduit, and which branch channel includes a filter.

According to an eighth seventh aspect of the present invention there may be provided a method of treatment of a patient, comprising:

lining the interior of a funnel having a first open end and a second open end with a liner of flexible material;

fitting the first open end of the funnel over the nose and/or mouth of the patient;

introducing medicament in aerosol form into the interior of the funnel through the second open end of said funnel;

supplying the medicament to the patient by inhalation of the aerosol by the patient from the interior of the funnel. Preferably, the method of the eighth aspect of the present invention further comprises the steps of restricting the flow of aerosol through the interior of the funnel when aerosol is introduced into the interior of the funnel, and subsequently permitting the flow of aerosol through the interior of the funnel to allow the medicament to be supplied to the patient from the funnel.

The present invention has been developed for the treatment of patients with a measles vaccine. However, the present invention is not limited to such vaccines, and the present invention may be used for other medicaments which can be supplied in aerosol form for use in an immunisation programme. The low cost nature of the present invention makes it particularly suitable for immunisation programmes in less developed countries.

Embodiments of the present invention will now be described in detail, by way of example, with reference to the accompanying drawings, in which:

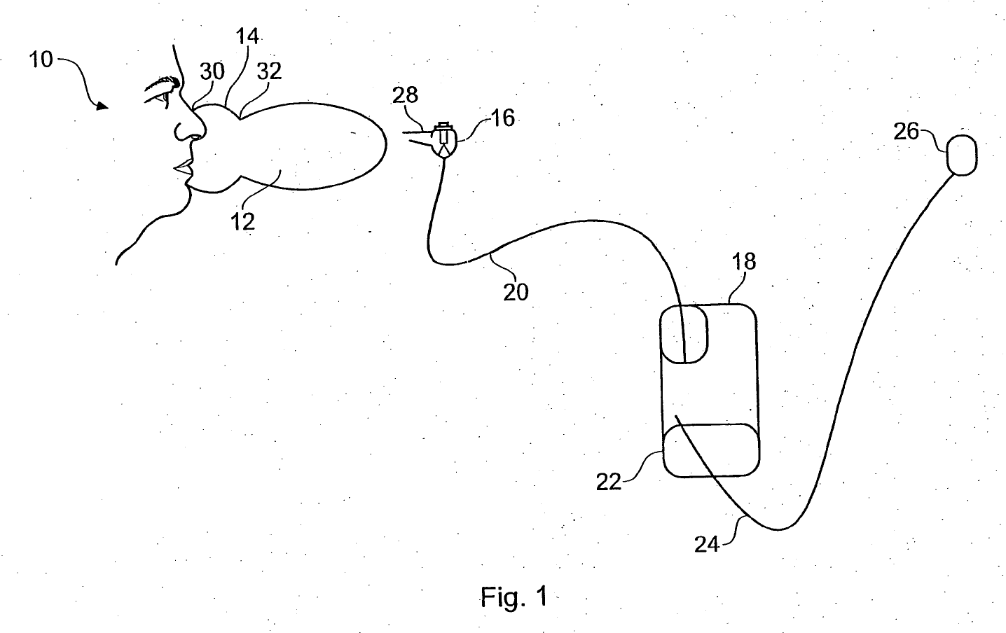

FIG. 1 is a general schematic view of the delivery of a medicament in accordance with the first to fourth aspects of the present invention;

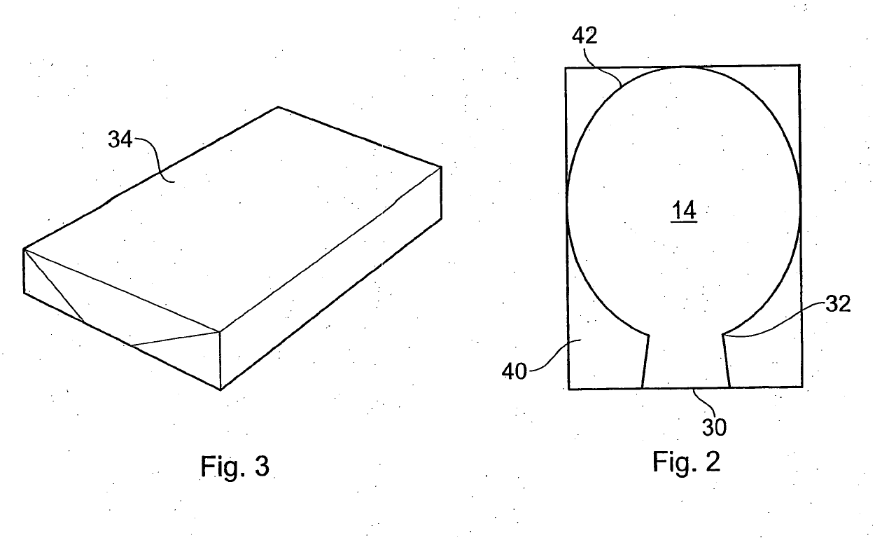

FIG. 2 illustrates the bag used in the present invention as part of a sheet;

FIG. 3 illustrates a block of such sheets;

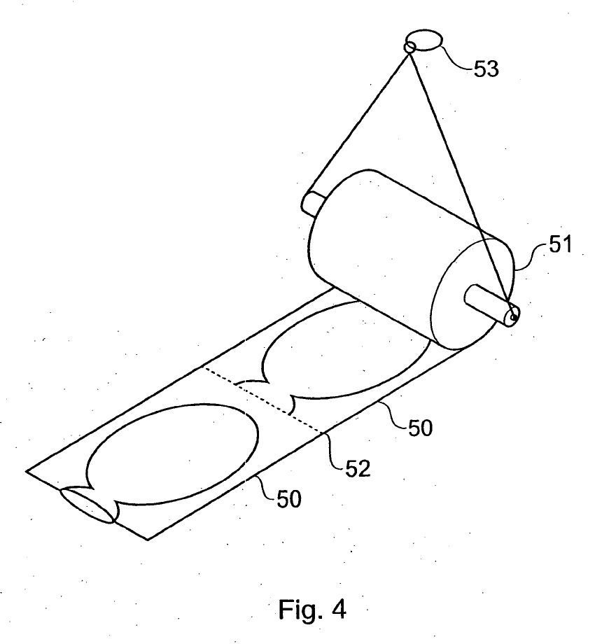

FIG. 4 illustrates a roll of such sheets on a bobbin dispenser;

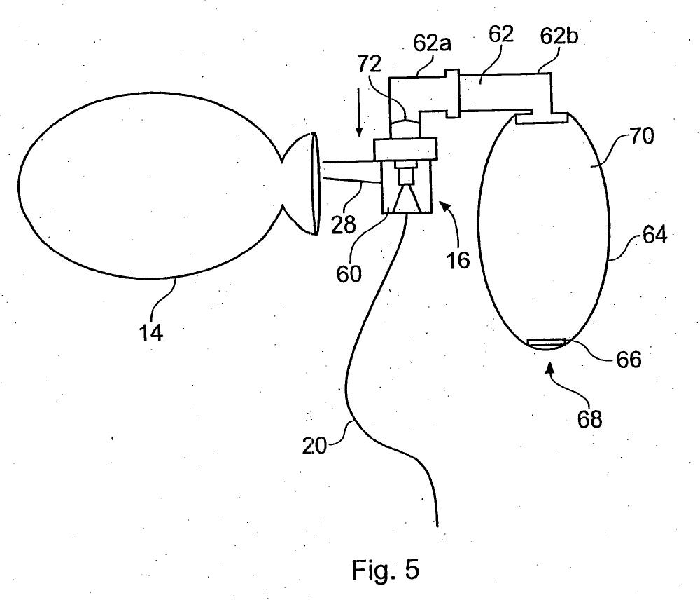

FIG. 5 illustrates a modification to the delivery arrangement of FIG. 1;

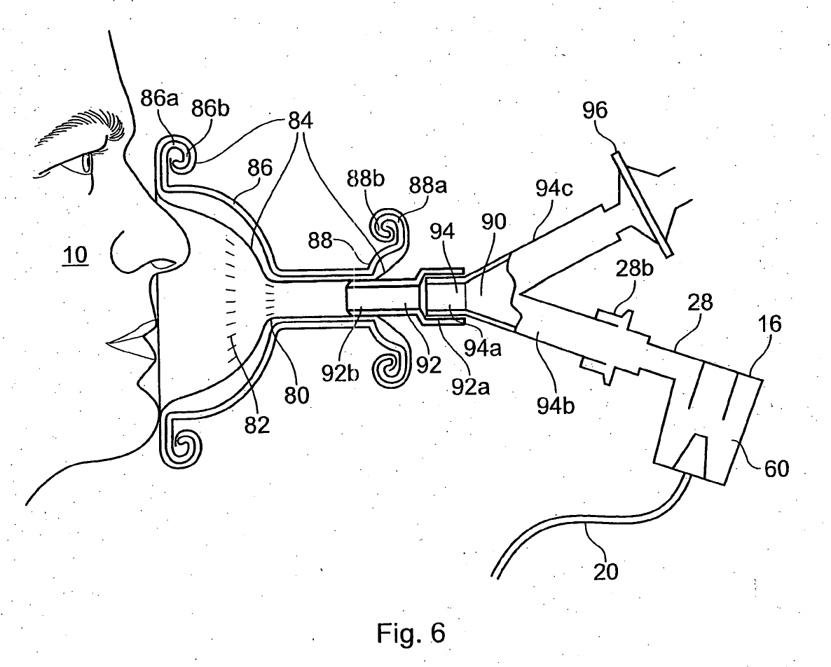

FIG. 6 is a general schematic view of an apparatus for delivery of a medicament in accordance with the fifth to eighth aspects of the present invention; and

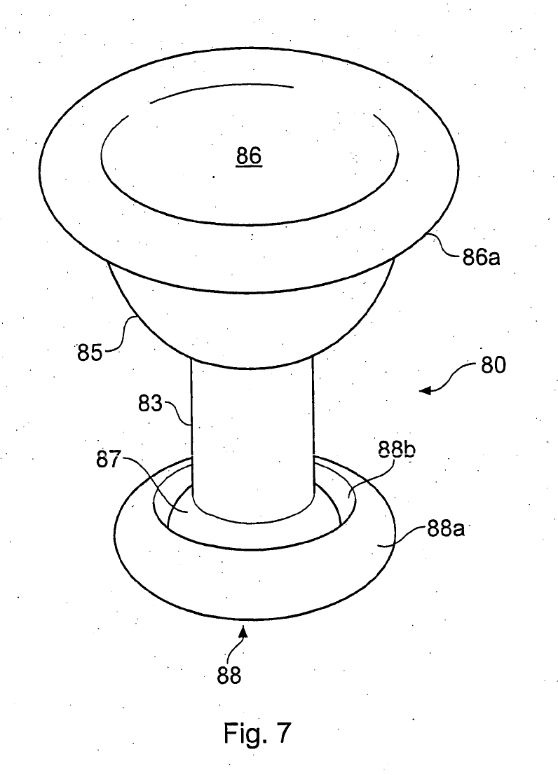

FIG. 7 is a detail of the apparatus of FIG. 6

Referring first to FIG. 1, a patient 10, is treated with a medicament, such as a measles vaccine, by inhaling the medicament in aerosol form from the interior 12 of a bag 14 of edible material. As illustrated in FIG. 1, the bag 14 is placed over the mouth and nose of the patient 10, so that the patient 10 breathes the air, and hence the aerosol in the air, from the interior of the bag 14, thereby ingesting the aerosol.

Such a delivery arrangement is of particular value when the patient is a child or infant, since they will readily be able to breathe the air and aerosol in the bag 14 but will not be frightened or distressed by having the bag 14 placed over their nose and mouth. Moreover, the bag 14 may then be disposed of, rather than being re-used with another patient to prevent cross-contamination.

As mentioned above, the bag 14 is of edible material and it is envisaged that ingestion of the bag 14 itself by the patient represents the primary way of disposal of the bag 14. The bag 14 may thus be made of rice paper, which is easily digestible, or may be made of a polymer of food-based material. The bag 14 may be eaten directly, or may be immersed in water, at least partially to dissolve it, prior to ingestion.

Since the bag 14 is intended to be ingested, it is preferably impregnated with vitamins and/or nutritional minerals, so that those vitamins and/or minerals will be absorbed by the patient. Thus the patient not only receives a dose of the medicament, but also a dietary supplement. A flavour can be imparted to the bag as a reward and encouragement to the patient to eat the bag.

In this embodiment, a nebuliser 16 is used to fill the bag. Alternative means of generating an aerosol may be used, e.g. a spray of compressed gas having the medicament mixed therein. The nebuliser 16 is powered by a compressor 18 producing a flow of compressed air to the nebuliser 16 via a low pressure compressed air line 20. The compressor 18 may have a battery 22 and/or a power line 24 terminating in a plug 26 for connection to an A/C power source. Indeed, the battery 22 may be re-chargeable from the A/C source to enable the nebuliser 16 to be used at sites remote from A/C power sources. A solar power system may also be used to drive the compressor 18, as may a DC 12V power source.

It is preferable that the medicament is supplied to the point of use in dry powder form, e.g. in a suitable phial. The dry powder medicament is then reconstituted at the point of use by a suitably trained person by adding a liquid dilutent of e.g. sterile water to produce a suitable volume of fluid containing the medicament.

The liquid medicament is then placed inside a pot of the nebuliser 16, and the nebuliser 16 is then driven by the compressor 18 to supply the vaccine to the interior of the bag 14. To do this the bag 14 is placed over the outlet 28 of the nebuliser 16 to cause the medicament, in aerosol form, to enter the hollow interior 12 of the bag 14. The compressor 18 may be operable to drive the nebuliser 16 for a measured period of time, which period of time is chosen to provide a suitable amount of aerosol into the bag 14. 30 seconds may be a suitable time.

To prevent escape of the aerosol from the bag 14 between the time in which the aerosol is supplied from the nebuliser 16 and the time in which the opening 30 of the bag is placed over the mouth and nose of the patient 10, the bag 14 needs to be closed. In practice, it is found that manual constriction of the bag 14 achieves sufficient sealing that there will be sufficient aerosol in the bag 14, assuming that the bag 14 is promptly transferred from the nebuliser 16 to the patient 10. To assist in this, the bag 14 preferably has a constriction 32 forming a neck, which can be clasped between the first and second fingers of the person transferring the bag 14 from the nebuliser 16 to the patient 10 (which may not be the patient themselves, particularly if the patient is a child). The constriction 32 is constricted over the outlet 28 of the nebuliser when the aerosol is supplied to the interior 12 of the bag 14, and then further constricted during transfer from the nebuliser 16 to the patient 10. Whilst more elaborate sealing arrangements are possible, they will make the bag more expensive to produce.

The pot of the nebuliser 16 is preferably disposable so that again there is no cross-contamination by e.g. re-filling the pot. The medicament is preferably a vaccine, more preferably a measles vaccine.

Once the bag 14 has been placed over the nose and mouth of the patient 10, the patient breathes in and out of the bag for e.g. 30 seconds, to inhale the aerosol. The bag will inflate and deflate whilst the patient 10 is breathing, thereby giving a visible indication that the patient is indeed inhaling the aerosol. This is particularly useful when the patient is a baby or child, who may not be able to give a verbal account of their behaviour.

Since the amount of aerosol required by a single patient is small, a suitable sized nebuliser pot will provide sufficient medicament for a large number of patients (40 or so). Thus, it is desirable for the bag 14 for each patient to be delivered to the treatment site in convenient form. Thus, for example, as shown in FIG. 2, the bag may be formed by a two-ply sheet 40 of edible material (e.g. rice paper), which sheet has a perforation 42 therein defining the periphery of the bag. The plies of the two-ply sheet 40 are sealed together immediately inside the perforation 42, so that the two plies form respective walls of the bag 14 when the bag 14 is released from the sheet 40. To release the bag 14, the sheet 40 is torn along the perforation 42, and the parts of the sheet 40 which do not correspond to the bag 14 can then be disposed of by eating.

FIG. 2 also shows clearly the constriction 32 forming the neck of the bag 14, and how the bag splays between the constriction 32 and the opening 30.

The sheet illustrated in FIG. 2 may be provided in a block 34 of such sheets, as illustrated in FIG. 3. Alternatively, a plurality of sheets, corresponding to sheet 40 shown in FIG. 2, may be supplied end-to-end, with perforations between each sheet, so that a sheet to be used can be torn off from the other sheets. Such end-to-end connection of sheets could enable the sheets to be provided in a roll, rather than in the block 34 of FIG. 3.

This arrangement is illustrated in FIG. 4.

FIG. 4 shows that plurality of sheets 50, which may be identical to the sheets 40 shown in FIG. 2, may be attached end to end on a roll 51. The sheets are separated by perforations 52. The roll may be suspended, as illustrated at 53.

Whether the sheets are provided in a roll so as in FIG. 4, or in a block 34 as illustrated in FIG. 3, it is preferable that they are supplied to the treatment site enclosed within an anti-contamination protection.

One potential problem with the embodiment of FIG. 1 is that the nebuliser 16 needs to have sufficient power to inflate the bag 14. If it does not, the bag 14 will not be correctly filled with aerosol medicament. To overcome this problem, the arrangement of FIG. 1 may be modified as shown in FIG. 5. In FIG. 5, parts which correspond to those in FIG. 1 are indicated by the same reference numerals. Note also that FIG. 5 shows the nebuliser 16 in more detail, more clearly illustrating the pot 60 of the nebuliser. Note also that the compressor 18, power line 24 and plug 26 are omitted in FIG. 5.

In the embodiment illustrated in FIG. 5, the nebuliser 16 is connected via a duct 62 to reservoir 64. The reservoir 64 terminates, at its end remote from the duct 62, in a one-way valve 66. Thus, air can be introduced in the direction shown by arrow 68 into the hollow interior 70 of the reservoir 64. In use, the reservoir 64 may be squeezed at the time of activation of the nebuliser 16, to provide an airflow from the reservoir 64 through the duct 62 to the nebuliser 16, then through the outlet 28 to the bag 14. That airflow assists in the inflation of the bag 14. To prevent passage of aerosol to the reservoir 64, a further one-way valve 72 may be provided in the duct 62. It is convenient if the duct 62 is formed to L-shaped parts 62a, 62b as shown in FIG. 5, so that both the nebuliser 16 and the reservoir 64 can be below the duct 62. The components of the duct 62 may be disposed of at the same time as the disposal of the nebuliser 16, again reducing the risk of cross-infection.

Referring to FIG. 6, a patient 10 is treated with a medicament, such as a measles vaccine, by inhaling the medicament in aerosol form from the interior 82 of a funnel 80 through a first open end 86 of the funnel 80. The funnel 80 is lined with an liner 84 of edible flexible material. The aerosol is created by a nebuliser 16 and is introduced to the interior 82 of the funnel 80 through a conduit 90, which is connected to a second open end 88 of the funnel 80 and communicates with a hollow duct 83, the interior of which forms the interior 82 and the ends of which communicate with splayed regions 85, 87 which terminate in the first and second open ends 86, 88 respectively.

As illustrated in FIG. 6, a first open end 86 of the funnel 80 is placed over the mouth and nose of the patient 10, so that patient 10 breathes air, and hence the aerosol in the air, from the interior 82 of the funnel 80, thereby ingesting the aerosol.

The liner 84 is arranged such that the patient's nose and mouth do not directly contact the interior 82 of the funnel 80, but instead contact the liner 84. The liner 84 therefore acts as a barrier between the interior 82 of the funnel 80 and the patient's body to prevent contamination of the funnel 80 by the patient 10.

In the embodiment of FIG. 6, the liner 84 also covers a rim 86a of the first open end 86 of the funnel 80. The rim 86a is bent back on itself to form a flange 86b, and the liner 84 further envelops the flange 86b. As the liner 84 envelops the whole of the first open end 86 of the funnel 80, this has the advantage that any contact of the first open end 86 of the funnel 80 with the patient 10 is more likely prevented. The second open end 88 of the funnel 80 also has a rim 88a, which is bent back on itself to form a flange 88b. The liner 84 envelops the flange 88b. Note that the funnel 80 is shown schematically in FIG. 6 and is shown in more detail in FIG. 7.

The ends of the liner 84 are tucked under flanges 86b and 88b at the first open end 86 and second open end 88 respectively of funnel 80, in order to hold the liner 84 in place, relative to the funnel 80. With this arrangement, on assembly of the device shown in FIG. 6, the liner 84 can be more easily arranged to line the interior 82 of the funnel 80 as it can be held at both ends of the funnel 80. Furthermore, the liner 84 can be held against the rim 88a or flange 88b such that when the first end of conduit 90 is inserted into the second open end 88 of the funnel 80 during assembly of the device, the liner 84 will not ruffle or block the interior 82 of the funnel 80. This leaves the interior 82 of the funnel 80 clear for the subsequent passage of aerosol through the funnel 80.

The rim 86a of the funnel 80 is broader than the rim 88a of the funnel 80, i.e. it has a greater diameter. With this feature, there is provided the option of reversing the funnel 80 to present the narrower open end of the funnel 80 to the patient 10, e.g. if the patient is a young child, should the first open end 86 (as shown) be too broad to mould suitably to the mouth and nose of the patient 10.

The conduit 90 comprises a connector 92 with a female socket part 92a and a male socket part 92a. The connector 92 is attached to the second open end 88 of the funnel 80 via the male socket part 92a. A male part 94 is attached to the connector 92. The parts of the conduit 90 are made from polyethylene and k resin ABS. Although it is preferable that the connector 92 and the male part 94 of the conduit 90 are made as one piece, in this embodiment the connector 92 and the male part 94 are formed separately. A first end of the connector 92 is attached directly to the second open end 88 of the funnel 80. A second end of the connector 92 has the female socket part 92a, and the male part 94 has a male end 94a which is plugged into the female socket part 92a of the female part 92. The result of this arrangement is that a first end of the conduit 90 is attached to the second open end 88 of the funnel 80, and a second end of the conduit 90 is attached to the nebuliser 16, via elastomeric connector 28b.

Interference fit ISO (International Organization for Standardisation) standard connectors are used at the interface of the funnel 80 and female part 92, and at the interface of the female part 92 and the male part 94.

In this embodiment, the internal diameter of the passageways through the conduit 90 is 22 mm. 22 mm is the ISO-required internal dimension for breathing systems for adults. (ISO 5367—Breathing tubes intended for use with anaesthetic apparatus and ventilators).

The 22 mm male part 92 may be inserted into either end of funnel 80, to provide devices suitable for use with both infants and older people.

Both the interface between the female part 92 and the funnel 80 and the interface between the female part 92 and the male part 94 are airtight, such that air flowing through the conduit 90 cannot leak from the inside of the conduit 90 to the outside of the conduit 90 at these interfaces. The funnel 80 is made from silicon rubber, making it flexible and elastic and so more suitable to provide an airtight joint with the conduit 90.

The conduit 90 has a branch channel which diverges from the conduit 90 between its first and second ends. The main channel of the conduit 90 is an inspiratory limb 94b and the branch channel is an expiratory limb 94c. A nebuliser 16 is attached to the main channel 94b via the outlet 28 of the nebuliser 16 and an airtight elastomeric connector 28b.

It is preferable that the medicament is supplied to the point of use in dry powder form, e.g. in a suitable phial. The dry powder medicament is then reconstituted at the point of use by a suitably trained person by adding a liquid dilutent of e.g. sterile water to produce a suitable volume of fluid containing the medicament. The liquid medicament is then placed inside a pot 60 of the nebuliser 16, and the nebuliser 16 is then driven by a compressor (not shown) to create an aerosol and supply the medicament to the interior 82 of the funnel 80. As mentioned above, in this embodiment the nebuliser 16 is powered by a compressor producing a flow of compressed air to the nebuliser 16 via a low pressure compressed air line 20.

As can be noted from FIG. 6, the funnel 80 has a central portion of reduced diameter compared to the diameter of the first and second open ends 86, 88. With this feature, the passageway through the interior 82 of the funnel 80 can be more easily closed by squeezing it between the open ends 86, 88 to prevent loss of a significant amount of the aerosol from the interior 82 of the funnel 80. This allows medicament to accumulate in the interior 82 of the funnel 80 such that, once the restriction is removed and the funnel 80 is placed over the nose and/or mouth of the patient 10, the patient 10 is able to inhale a suitable dose of the vaccine from the interior 82 of the funnel 80.

When a patient 10 is using the device shown in FIG. 6, the branch channel 94c provides an exhaust for air expelled from the patient 10 as they breath out. A filter 96 is provided in the branch channel 94c. When the patient 10 breathes in, he/she will inhale air from the nebuliser through the main channel 94b, and air from the branch channel 94c. When the patient breathes out, filter 96 filters expired breath to reduce the chance of contaminating a nurse administering the vaccine to the patient 10.

After use of the device shown in FIG. 6, the edible liner 84 is removed from the funnel 80 and is disposed of, leaving the funnel 80 uncontaminated and available for reuse with subsequent patients. This reduces the cost of a mass-immunisation program as only the liner 84, and not the funnel 80, need to be replaced for use with different patients. The components of the conduit 90 may be disposed of at the same time as the disposal of the liner 84, again reducing the risk of cross-infection.

As the liner 84 is of edible material, it can be ingested readily by the patient 10, which provides a means of disposal of the liner 84 and so prevents cross-contamination from one patient to another. To achieve such an edible liner 84, in this embodiment the liner 84 is made of a food-derived polymer. Moreover, the liner 84 is impregnated with vitamins and/or nutritional minerals, so that those vitamins and/or nutritional minerals are absorbed by the patient when the liner 84 is ingested. This permits the liner 84 to serve a second purpose after the medicament has been ingested.

As will be appreciated from the above, the components needed for treatment of patients are simple, and thus may be provided at low cost. This is particularly of value where the invention is to be used in less developed countries. The bag or liner is simple to produce in bulk and the edible nature of the bag provides a ready, and acceptable method of disposal. The impregnation of the bag or liner with vitamins and/or nutritional minerals and/or with a flavouring which encourages the patient to consume the bag or liner (or more probably the parent or other carer of the patient to allow the patient to consume the bag or liner), rather than putting the bag to some other use, means that bags or liners are unlikely to be used to supply medicament to multiple patients, thereby reducing the risk of cross-contamination or environmental waste.

Furthermore, as the funnel is reversible, with different diameters of rim at either end which can be introduced to the face of a patient, a reduction is achieved in the number of components required to be carried during a mass-immunisation program. Patients of various ages, i.e. each with different dimensions of noses and mouths, can be treated using a single, flexible, reversible funnel.

1. A kit of parts for use in delivery of a medicament to a patient, comprising:
at least one bag of flexible edible material,
a container for said medicament; and
means for generating an aerosol of at least part of said medicament in said container for supply of said aerosol to the interior of the bag.

2. A kit of parts according to claim 1, wherein the bag is of a food-derived polymer.

3. A kit of parts according to claim 1, wherein the bag is part of a sheet, the sheet being perforated to permit separation of the bag from the rest of the sheet.

4. A kit of parts according to claim 3, wherein said sheet is one of a plurality of identical sheets.

5. A kit of parts according to any one of claims 1, wherein the bag is impregnated with vitamins and/or flavouring and/or nutritional minerals.

6. A kit of parts according to any one of claims 1, wherein the bag has a constricted neck, and is splayed between said neck and an opening for fitting over the nose and/or mouth of said patient.

7. A kit of parts according to any one of claims 1, wherein said means for generating an aerosol is a nebuliser.

8. A kit of parts according to claim 7, further including a compressor for driving said nebuliser.

9. A medicament delivery device comprising a bag of flexible edible material, and a medicament in aerosol form in said bag, wherein said bag is closed but is openable to fit over the nose and/or mouth of a patient.

10. A medicament delivery device according to claim 9, wherein the bag is of rice paper.

11. A medicament delivery device according to claim 9, wherein the bag is of a food-derived polymer.

12. A medicament delivery device according to any one of claims 9, wherein the bag is impregnated with vitamins and/or flavouring and/or nutritional minerals.

13. A medicament delivery device according to any one of claims 9, wherein the bag has a constricted neck, and is splayed between said neck and an opening for fitting over the nose and/or mouth of said patient.

14-21. (canceled)

22. A device for use in delivery of a medicament to a patient, comprising:
a funnel having a first open end and a second open end,
a liner of flexible material, said liner lining the interior of said funnel,
a container for said medicament,
an apparatus for generating an aerosol of at least part of said medicament in said container, and
a conduit arranged between said apparatus for generating an aerosol and said second open end of said funnel, thereby to permit said aerosol to be supplied from said apparatus to the interior of said funnel via said second open end, for inhalation by the patient via said first open end.

23. A device according to claim 22 wherein said funnel is made of flexible material.

24. A device according to claim 22, wherein said liner covers a rim of the first open end of said funnel.

25. A device according to any one of claims 22, wherein said liner covers a rim of said second open end of said funnel.

26. A device according to any one of claims 22, wherein the rim of the first open end of said funnel is flanged, such that the rim is bent back on itself, and said liner envelops said flange.

27. A device according to any one of claims 22, wherein the rim of the second open end of said funnel is flanged, such that the rim is bent back on itself, and said liner envelops said flange.

28. A device according to any one of claims 22, wherein the liner is of edible material.

29. A device according to any one of claims 22, wherein the liner is of rice paper.

30. A device according to any one of claims 22, wherein the liner is of a food-derived polymer.

31. A device according to any one of claims 22, wherein the liner is impregnated with vitamins and/or flavouring and/or nutritional minerals.

32. A device according to any one of claims 22, wherein the diameter of a rim of the first open end of the funnel is greater than that of a rim of the second open end of the funnel.

33. A device according to any one of claims 22, wherein the diameter of a rim of the second open end of the funnel is greater than that of a rim of the first open end of the funnel.

34. A device according to any one of claims 22, wherein said conduit includes a branch channel, diverging from the conduit between said apparatus and said second open end of said funnel, which branch channel includes a filter.

35. A device according to any one of claims 22, wherein said apparatus for generating an aerosol is a nebuliser.

36. A device according to claim 35, further including a compressor for driving said nebuliser.

37. A kit of parts for use in delivery of a medicament to a patient, comprising:
a funnel having a first open end and a second open end,
a liner of flexible material, said liner being adapted to line the interior of said funnel,
a container for said medicament,
an apparatus for generating an aerosol of at least part of said medicament in said container, said apparatus adapted to supply the aerosol to the interior of the funnel via said second open end, and
a conduit adapted to be connectable by a first end to said second open end of said funnel and connectable by a second end to said apparatus for generating an aerosol.

38. A kit of parts according to claim 37, wherein said conduit includes a branch channel, which branch channel branches from said conduit between said first and second ends of said conduit, and which branch channel includes a filter.

39. A kit of parts according to claim 37, wherein said apparatus for generating an aerosol is a nebuliser.

40. A kit of parts according to claim 39, further including a compressor for driving said nebuliser.

41. A kit of parts according to any one of claims 37, wherein the wider of the two open ends of said funnel is adapted to be connectable to said conduit, and thus forms the second open end of the funnel.

42. A kit of parts according to any one of claims 37, wherein the narrower of the two open ends of said funnel is adapted to be connectable to said conduit, and thus forms the second open end of the funnel.

43. A kit of parts according to any one of claims 37, wherein the liner is part of a sheet, the sheet being perforated to permit separation of the liner from the rest of the sheet.

44. A kit of parts according to claim 43, wherein said sheet is one of a plurality of identical sheets.

45. A method of assembling a device for delivery of a medicament to a patient, said device comprising:
a funnel having a first open end and a second open end,
a liner of flexible material, said liner being adapted to line the interior of said funnel,
a container for said medicament, and
an apparatus for generating an aerosol of at least part of said medicament in said container to supply the aerosol to the interior of the funnel via said second open end,
said method including the steps of:
arranging the liner in the funnel such that the liner lines the interior of the funnel,
connecting the second open end of said funnel to said apparatus for generating an aerosol such that they are in fluid communication, and
connecting said apparatus for generating an aerosol to said container such that they are in fluid communication.

46. A method according to claim 45, wherein the step of arranging the liner in the funnel such that the liner lines the interior of the funnel is carried out before the step of connecting the second open end of said funnel to said apparatus for generating an aerosol.

47. A method according to claim 45, wherein the step of arranging the liner in the funnel such that the liner lines the interior of the funnel is carried out after the step of connecting the second open end of said funnel to said apparatus for generating an aerosol.

48. A method according to any one of claims 45, wherein the liner is of edible material.

49. A method according to any one of claims 45, wherein when said liner is arranged to line the interior of the funnel it also covers a rim of the first open end of said funnel.

50. A method according to any one of claims 45, wherein when said liner is arranged to line the interior of the funnel it also covers a rim of said second open end of said funnel.

51. A method according to any one of claims 45, wherein the rim of the first open end of said funnel is flanged, such that the rim is bent back on itself, and when said liner is arranged to line the interior of the funnel it also envelops said flange.

52. A method according to any one of claims 45, wherein the rim of the second open end of said funnel is flanged, such that the rim is bent back on itself, and when said liner is arranged to line the interior of the funnel it also envelops said flange.

53. A method according to any of claims 45, wherein the step of connecting the second open end of said funnel to said apparatus for generating an aerosol includes the steps of:
connecting the second open end of said funnel to a first end of a conduit, and
connecting said apparatus for generating an aerosol to a second end of said conduit,
wherein said conduit includes a branch channel, which branches from said conduit between the first and second ends of said conduit, and which branch channel includes a filter.

54-55. (canceled)
